SEQUENCE LISTING (99 sequences)

SEQ ID NO: 1 - DNA, 12 bp, unidentified
  misc_feature 1..12: note = Artificial sequence
  source 1..12: mol_type = other DNA
ctcgagtagcag

SEQ ID NO: 2 - DNA, 12 bp, unidentified
  misc_feature 1..12: note = Artificial sequence
  source 1..12: mol_type = other DNA
cagcacacgtca

SEQ ID NO: 3 - DNA, 12 bp, unidentified
  misc_feature 1..12: note = Artificial sequence
  source 1..12: mol_type = other DNA
acagcgatcgac

SEQ ID NO: 4 - DNA, 12 bp, unidentified
  misc_feature 1..12: note = Artificial sequence
  source 1..12: mol_type = other DNA
tagtgtacgaca

SEQ ID NO: 5 - DNA, 12 bp, unidentified
  misc_feature 1..12: note = Artificial sequence
  source 1..12: mol_type = other DNA
tagtcgtctagc

SEQ ID NO: 6 - DNA, 12 bp, unidentified
  misc_feature 1..12: note = Artificial sequence
  source 1..12: mol_type = other DNA
catcagctgcac

SEQ ID NO: 7 - DNA, 12 bp, unidentified
  misc_feature 1..12: note = Artificial sequence
  source 1..12: mol_type = other DNA
tagtagcacgca

SEQ ID NO: 8 - DNA, 12 bp, unidentified
  misc_feature 1..12: note = Artificial sequence
  source 1..12: mol_type = other DNA
cagtcagctgac

SEQ ID NO: 9 - DNA, 12 bp, unidentified
  misc_feature 1..12: note = Artificial sequence
  source 1..12: mol_type = other DNA
cagcagtctacg

SEQ ID NO: 10 - DNA, 12 bp, unidentified
  misc_feature 1..12: note = Artificial sequence
  source 1..12: mol_type = other DNA
cagctagagcac

SEQ ID NO: 11 - DNA, 12 bp, unidentified
  misc_feature 1..12: note = Artificial sequence
  source 1..12: mol_type = other DNA
ctagcatgacga

SEQ ID NO: 12 - DNA, 12 bp, unidentified
  misc_feature 1..12: note = Artificial sequence
  source 1..12: mol_type = other DNA
actctacgcgac

SEQ ID NO: 13 - DNA, 12 bp, unidentified
  misc_feature 1..12: note = Artificial sequence
  source 1..12: mol_type = other DNA
ctgtcgagctga

SEQ ID NO: 14 - DNA, 12 bp, unidentified
  misc_feature 1..12: note = Artificial sequence
  source 1..12: mol_type = other DNA
acagacgagtca

SEQ ID NO: 15 - DNA, 12 bp, unidentified
  misc_feature 1..12: note = Artificial sequence
  source 1..12: mol_type = other DNA
ctatgatctacg

SEQ ID NO: 16 - DNA, 12 bp, unidentified
  misc_feature 1..12: note = Artificial sequence
  source 1..12: mol_type = other DNA
ctcagagcagac

SEQ ID NO: 17 - DNA, 12 bp, unidentified
  misc_feature 1..12: note = Artificial sequence
  source 1..12: mol_type = other DNA
acagagactacg

SEQ ID NO: 18 - DNA, 12 bp, unidentified
  misc_feature 1..12: note = Artificial sequence
  source 1..12: mol_type = other DNA
ctctgcactagc

SEQ ID NO: 19 - DNA, 12 bp, unidentified
  misc_feature 1..12: note = Artificial sequence
  source 1..12: mol_type = other DNA
actagtgacgac

SEQ ID NO: 20 - DNA, 12 bp, unidentified
  misc_feature 1..12: note = Artificial sequence
  source 1..12: mol_type = other DNA
tacgatgcgtac

SEQ ID NO: 21 - DNA, 12 bp, unidentified
  misc_feature 1..12: note = Artificial sequence
  source 1..12: mol_type = other DNA
acgagacatcac

SEQ ID NO: 22 - DNA, 12 bp, unidentified
  misc_feature 1..12: note = Artificial sequence
  source 1..12: mol_type = other DNA
catcactgcaca

SEQ ID NO: 23 - DNA, 12 bp, unidentified
  misc_feature 1..12: note = Artificial sequence
  source 1..12: mol_type = other DNA
ctgacatcacag

SEQ ID NO: 24 - DNA, 12 bp, unidentified
  misc_feature 1..12: note = Artificial sequence
  source 1..12: mol_type = other DNA
tagtacgactac

SEQ ID NO: 25 - DNA, 12 bp, unidentified
  misc_feature 1..12: note = Artificial sequence
  source 1..12: mol_type = other DNA
cacgcagagtca

SEQ ID NO: 26 - DNA, 12 bp, unidentified
  misc_feature 1..12: note = Artificial sequence
  source 1..12: mol_type = other DNA
cacacgcatagc

SEQ ID NO: 27 - DNA, 12 bp, unidentified
  misc_feature 1..12: note = Artificial sequence
  source 1..12: mol_type = other DNA
acgtatgtctag

SEQ ID NO: 28 - DNA, 12 bp, unidentified
  misc_feature 1..12: note = Artificial sequence
  source 1..12: mol_type = other DNA
catctcactaga

SEQ ID NO: 29 - DNA, 12 bp, unidentified
  misc_feature 1..12: note = Artificial sequence
  source 1..12: mol_type = other DNA
atcgtcatacga

SEQ ID NO: 30 - DNA, 12 bp, unidentified
  misc_feature 1..12: note = Artificial sequence
  source 1..12: mol_type = other DNA
tctagcacgtgc

SEQ ID NO: 31 - DNA, 12 bp, unidentified
  misc_feature 1..12: note = Artificial sequence
  source 1..12: mol_type = other DNA
tcagactgtcac

SEQ ID NO: 32 - DNA, 12 bp, unidentified
  misc_feature 1..12: note = Artificial sequence
  source 1..12: mol_type = other DNA
tcagtagtctac

SEQ ID NO: 33 - DNA, 12 bp, unidentified
  misc_feature 1..12: note = Artificial sequence
  source 1..12: mol_type = other DNA
tactgacacgac

SEQ ID NO: 34 - DNA, 12 bp, unidentified
  misc_feature 1..12: note = Artificial sequence
  source 1..12: mol_type = other DNA
catactcatgag

SEQ ID NO: 35 - DNA, 12 bp, unidentified
  misc_feature 1..12: note = Artificial sequence
  source 1..12: mol_type = other DNA
cacatgcagtcg

SEQ ID NO: 36 - DNA, 12 bp, unidentified
  misc_feature 1..12: note = Artificial sequence
  source 1..12: mol_type = other DNA
cacagatcgagc

SEQ ID NO: 37 - DNA, 12 bp, unidentified
  misc_feature 1..12: note = Artificial sequence
  source 1..12: mol_type = other DNA
tcgtgactcagc

SEQ ID NO: 38 - DNA, 12 bp, unidentified
  misc_feature 1..12: note = Artificial sequence
  source 1..12: mol_type = other DNA
cagcacgatagc

SEQ ID NO: 39 - DNA, 12 bp, unidentified
  misc_feature 1..12: note = Artificial sequence
  source 1..12: mol_type = other DNA
ctacagcacacg

SEQ ID NO: 40 - DNA, 12 bp, unidentified
  misc_feature 1..12: note = Artificial sequence
  source 1..12: mol_type = other DNA
ctgtacgcatgc

SEQ ID NO: 41 - DNA, 12 bp, unidentified
  misc_feature 1..12: note = Artificial sequence
  source 1..12: mol_type = other DNA
ctacacacagcg

SEQ ID NO: 42 - DNA, 12 bp, unidentified
  misc_feature 1..12: note = Artificial sequence
  source 1..12: mol_type = other DNA
actcgtgcgaga

SEQ ID NO: 43 - DNA, 12 bp, unidentified
  misc_feature 1..12: note = Artificial sequence
  source 1..12: mol_type = other DNA
tagcgcatgcac

SEQ ID NO: 44 - DNA, 12 bp, unidentified
  misc_feature 1..12: note = Artificial sequence
  source 1..12: mol_type = other DNA
acgagcatcgca

SEQ ID NO: 45 - DNA, 12 bp, unidentified
  misc_feature 1..12: note = Artificial sequence
  source 1..12: mol_type = other DNA
ctcgcgtacaca

SEQ ID NO: 46 - DNA, 12 bp, unidentified
  misc_feature 1..12: note = Artificial sequence
  source 1..12: mol_type = other DNA
ctgtagcatcgc

SEQ ID NO: 47 - DNA, 12 bp, unidentified
  misc_feature 1..12: note = Artificial sequence
  source 1..12: mol_type = other DNA
tctacgtatcgc

SEQ ID NO: 48 - DNA, 12 bp, unidentified
  misc_feature 1..12: note = Artificial sequence
  source 1..12: mol_type = other DNA
ctgagctgtaca

SEQ ID NO: 49 - DNA, 12 bp, unidentified
  misc_feature 1..12: note = Artificial sequence
  source 1..12: mol_type = other DNA
catgcacacaga

SEQ ID NO: 50 - DNA, 12 bp, unidentified
  misc_feature 1..12: note = Artificial sequence
  source 1..12: mol_type = other DNA
tcatcgacatag

SEQ ID NO: 51 - DNA, 12 bp, unidentified
  misc_feature 1..12: note = Artificial sequence
  source 1..12: mol_type = other DNA
cacacactgcga

SEQ ID NO: 52 - DNA, 12 bp, unidentified
  misc_feature 1..12: note = Artificial sequence
  source 1..12: mol_type = other DNA
cactgctagaca

SEQ ID NO: 53 - DNA, 12 bp, unidentified
  misc_feature 1..12: note = Artificial sequence
  source 1..12: mol_type = other DNA
acgcgtagctac

SEQ ID NO: 54 - DNA, 12 bp, unidentified
  misc_feature 1..12: note = Artificial sequence
  source 1..12: mol_type = other DNA
cacgtctatcgc

SEQ ID NO: 55 - DNA, 12 bp, unidentified
  misc_feature 1..12: note = Artificial sequence
  source 1..12: mol_type = other DNA
cagcatacacgc

SEQ ID NO: 56 - DNA, 12 bp, unidentified
  misc_feature 1..12: note = Artificial sequence
  source 1..12: mol_type = other DNA
cacagtcgacac

SEQ ID NO: 57 - DNA, 12 bp, unidentified
  misc_feature 1..12: note = Artificial sequence
  source 1..12: mol_type = other DNA
acgacgcgtaga

SEQ ID NO: 58 - DNA, 12 bp, unidentified
  misc_feature 1..12: note = Artificial sequence
  source 1..12: mol_type = other DNA
atgcgacagacg

SEQ ID NO: 59 - DNA, 12 bp, unidentified
  misc_feature 1..12: note = Artificial sequence
  source 1..12: mol_type = other DNA
atctactagtgc

SEQ ID NO: 60 - DNA, 12 bp, unidentified
  misc_feature 1..12: note = Artificial sequence
  source 1..12: mol_type = other DNA
atcatgagcaga

SEQ ID NO: 61 - DNA, 12 bp, unidentified
  misc_feature 1..12: note = Artificial sequence
  source 1..12: mol_type = other DNA
catcatagtgac

SEQ ID NO: 62 - DNA, 12 bp, unidentified
  misc_feature 1..12: note = Artificial sequence
  source 1..12: mol_type = other DNA
ctcgacagctca

SEQ ID NO: 63 - DNA, 12 bp, unidentified
  misc_feature 1..12: note = Artificial sequence
  source 1..12: mol_type = other DNA
acgctatcgagc

SEQ ID NO: 64 - DNA, 12 bp, unidentified
  misc_feature 1..12: note = Artificial sequence
  source 1..12: mol_type = other DNA
catgcgtctcag

SEQ ID NO: 65 - DNA, 12 bp, unidentified
  misc_feature 1..12: note = Artificial sequence
  source 1..12: mol_type = other DNA
actgtagacagc

SEQ ID NO: 66 - DNA, 12 bp, unidentified
  misc_feature 1..12: note = Artificial sequence
  source 1..12: mol_type = other DNA
tcgctgcatagc

SEQ ID NO: 67 - DNA, 12 bp, unidentified
  misc_feature 1..12: note = Artificial sequence
  source 1..12: mol_type = other DNA
cagtagcgtgag

SEQ ID NO: 68 - DNA, 12 bp, unidentified
  misc_feature 1..12: note = Artificial sequence
  source 1..12: mol_type = other DNA
actgtctgtgca

SEQ ID NO: 69 - DNA, 12 bp, unidentified
  misc_feature 1..12: note = Artificial sequence
  source 1..12: mol_type = other DNA
acgacagcatgc

SEQ ID NO: 70 - DNA, 12 bp, unidentified
  misc_feature 1..12: note = Artificial sequence
  source 1..12: mol_type = other DNA
ctctacatagac

SEQ ID NO: 71 - DNA, 12 bp, unidentified
  misc_feature 1..12: note = Artificial sequence
  source 1..12: mol_type = other DNA
cagcgagtgaca

SEQ ID NO: 72 - DNA, 12 bp, unidentified
  misc_feature 1..12: note = Artificial sequence
  source 1..12: mol_type = other DNA
cacgacatcagc

SEQ ID NO: 73 - DNA, 12 bp, unidentified
  misc_feature 1..12: note = Artificial sequence
  source 1..12: mol_type = other DNA
tcatgacgagag

SEQ ID NO: 74 - DNA, 12 bp, unidentified
  misc_feature 1..12: note = Artificial sequence
  source 1..12: mol_type = other DNA
acatcgtagtcg

SEQ ID NO: 75 - DNA, 12 bp, unidentified
  misc_feature 1..12: note = Artificial sequence
  source 1..12: mol_type = other DNA
ctagagatgcga

SEQ ID NO: 76 - DNA, 12 bp, unidentified
  misc_feature 1..12: note = Artificial sequence
  source 1..12: mol_type = other DNA
tatcagacatcg

SEQ ID NO: 77 - DNA, 12 bp, unidentified
  misc_feature 1..12: note = Artificial sequence
  source 1..12: mol_type = other DNA
acactatgcaca

SEQ ID NO: 78 - DNA, 12 bp, unidentified
  misc_feature 1..12: note = Artificial sequence
  source 1..12: mol_type = other DNA
cagagtagtgcg

SEQ ID NO: 79 - DNA, 12 bp, unidentified
  misc_feature 1..12: note = Artificial sequence
  source 1..12: mol_type = other DNA
catgactagtcg

SEQ ID NO: 80 - DNA, 12 bp, unidentified
  misc_feature 1..12: note = Artificial sequence
  source 1..12: mol_type = other DNA
ctacgtatatgc

SEQ ID NO: 81 - DNA, 12 bp, unidentified
  misc_feature 1..12: note = Artificial sequence
  source 1..12: mol_type = other DNA
ctacgctcgtag

SEQ ID NO: 82 - DNA, 12 bp, unidentified
  misc_feature 1..12: note = Artificial sequence
  source 1..12: mol_type = other DNA
ctcgtagctaga

SEQ ID NO: 83 - DNA, 12 bp, unidentified
  misc_feature 1..12: note = Artificial sequence
  source 1..12: mol_type = other DNA
ctactagacgca

SEQ ID NO: 84 - DNA, 12 bp, unidentified
  misc_feature 1..12: note = Artificial sequence
  source 1..12: mol_type = other DNA
tcatgctcgcga

SEQ ID NO: 85 - DNA, 12 bp, unidentified
  misc_feature 1..12: note = Artificial sequence
  source 1..12: mol_type = other DNA
acacgtagtgca

SEQ ID NO: 86 - DNA, 12 bp, unidentified
  misc_feature 1..12: note = Artificial sequence
  source 1..12: mol_type = other DNA
actgagcagcga

SEQ ID NO: 87 - DNA, 12 bp, unidentified
  misc_feature 1..12: note = Artificial sequence
  source 1..12: mol_type = other DNA
tcacgcgtagca

SEQ ID NO: 88 - DNA, 12 bp, unidentified
  misc_feature 1..12: note = Artificial sequence
  source 1..12: mol_type = other DNA
tcatgcactgcg

SEQ ID NO: 89 - DNA, 12 bp, unidentified
  misc_feature 1..12: note = Artificial sequence
  source 1..12: mol_type = other DNA
actgctatctcg

SEQ ID NO: 90 - DNA, 12 bp, unidentified
  misc_feature 1..12: note = Artificial sequence
  source 1..12: mol_type = other DNA
cagactctgacg

SEQ ID NO: 91 - DNA, 12 bp, unidentified
  misc_feature 1..12: note = Artificial sequence
  source 1..12: mol_type = other DNA
acacgcgatacg

SEQ ID NO: 92 - DNA, 12 bp, unidentified
  misc_feature 1..12: note = Artificial sequence
  source 1..12: mol_type = other DNA
tcgacgatgaca

SEQ ID NO: 93 - DNA, 12 bp, unidentified
  misc_feature 1..12: note = Artificial sequence
  source 1..12: mol_type = other DNA
atacatcgacga

SEQ ID NO: 94 - DNA, 12 bp, unidentified
  misc_feature 1..12: note = Artificial sequence
  source 1..12: mol_type = other DNA
cacatcactgac

SEQ ID NO: 95 - DNA, 12 bp, unidentified
  misc_feature 1..12: note = Artificial sequence
  source 1..12: mol_type = other DNA
tcagcatactca

SEQ ID NO: 96 - DNA, 12 bp, unidentified
  misc_feature 1..12: note = Artificial sequence
  source 1..12: mol_type = other DNA
tatcgtcgcacg

SEQ ID NO: 97 - DNA, 21 bp, unidentified
  misc_feature 1..21: note = Artificial sequence
  source 1..21: mol_type = other DNA
ctacacgacgctcttccgatc

SEQ ID NO: 98 - skipped

SEQ ID NO: 99 - DNA, 32 bp, unidentified
  misc_feature 1..32: note = Artificial sequence
  misc_difference 31: note = v is any nucleotide selected from A, C and G.
  misc_difference 32: note = n is a, c, g, or t
  source 1..32: mol_type = other DNA
ttttttttttttttttttttttttttttttvn

(19) United States
(12) Patent Application Publication
(10) Pub. No.: US 2024/0384336 A1
(43) Pub. Date: Nov. 21, 2024

(54) Optimized Set Of Oligonucleotides For Bulk RNA Barcoding And Sequencing

(71) Applicant: ECOLE POLYTECHNIQUE FEDERALE DE LAUSANNE (EPFL), Lausanne (CH)

(72) Inventors: Riccardo DAINESE, Cossonay-Ville (CH); Daniel ALPERN, Evian-Les-bains (FR); Vincent GARDEUX, Evian-les-Bains (FR); Bart DEPLANCKE, Cugy (CH)

(21) Appl. No.: 18/682,971

(22) PCT Filed: Aug. 23, 2022
(86) PCT No.: PCT/EP2022/073458

§ 371 Date: Feb. 12, 2024

(30) Foreign Application Priority Data
Aug. 24, 2021 (WO) PCT/EP2021/073346
Jun. 7, 2022 (WO) PCT/EP2022/065323

(51) Int. Cl. C12Q 1/6853; C12N 15/10; C12Q 1/6806

(52) U.S. Cl. CPC C12Q 1/6853; C12N 15/1096; C12Q 1/6806

(57) ABSTRACT

The present invention relates generally to the field of nucleic acid sequencing and provides oligonucleotide molecules and barcodes contained therein. These oligonucleotide molecules and barcodes molecules are useful in sequencing to identify and resolve errors.

### PRIORITY STATEMENT

This application is a national stage application under 35 U.S.C. § 371 of PCT International Application No. PCT/EP2022/073458, which has an international filing date of 23 Aug. 2022 and claims priority under 35 U.S.C. § 119 to International Application No. PCT/EP2022/065323 filed on 7 Jun. 2022 and International Application No. PCT/EP2021/073346 filed on 24 Aug. 2021. The contents of each application recited above are incorporated herein by reference in their entirety.

### REFERENCE TO A SEQUENCE LISTING

This application contains references to amino acid sequences and/or nucleic acid sequences which have been submitted concurrently herewith as the sequence listing .XML file entitled “ST26_SL_21_Feb_2024.xml”, file size 121 KiloBytes (KB), created on 21 Feb. 2024. The aforementioned sequence listing is hereby incorporated by reference in its entirety.

### FIELD OF THE INVENTION

The present invention relates generally to the field of nucleic acid sequencing and provides oligonucleotide molecules and barcodes contained therein. These oligonucleotide molecules and barcodes molecules are useful in sequencing to identify and resolve errors.

### BACKGROUND OF THE INVENTION

RNA sequencing has become the method of choice for genome-wide transcriptomic analyses as its price has substantially decreased over the last years. Nevertheless, the high cost of standard RNA library preparation and the complexity of the underlying data analysis still prevent this approach from becoming as routine as quantitative PCR (qPCR), especially when many samples need to be analyzed.

To alleviate this high cost, the emerging single-cell transcriptomics field implemented the sample barcoding/early multiplexing principle. This reduces both the RNA-seq cost and preparation time by allowing the generation of a single sequencing library that contains multiple distinct samples/cells (Ziegenhain et al., 2017, Mol. Cell 65, 631-643.e4).

Such a strategy could also be of value to reduce the cost and processing time of bulk

RNA sequencing of large sets of samples (Kilpinen et al., 2013, Science 342, 744-747; Waszak. et al., 2015, Cell 162, 1039-1050; Pradhan et al. 2017, Sci. Rep. 7, 42130). However, there have been surprisingly few efforts to explicitly adapt and validate the early-stage multiplexing protocols for reliable and affordable profiling of bulk RNA samples.

Early multiplexing protocols designed for single-cell RNA profiling (CEL-seq2, SCRB-seq, and STRT-seq) provide a great capacity for transforming large sets of samples into a unique sequencing library (Hashimshony et al. 2016, Genome Biol., 17, 77; Islam et al., 2012, Nat. Protoc. 7, 813-828; Soumillonet al., 2014, bioRxiv, 003236, doi:10.1101/003236). This is achieved by introducing a sample-specific barcode during the RT reaction using a “molecular tag” carried by either the oligo-dT or the template switch oligo (TSO). After individual samples have been “tagged”, they are pooled together, and the remaining steps are performed in bulk, thus shortening the time and cost of library preparation.

Since the tag is introduced to the terminal part of the transcript prior to fragmentation, the reads solely cover the 3′ or 5′ end of the transcripts. The 3′DGE approach for bulk RNA profiling, has been adopted in several recent studies, such as PLATE-seq (Bush et al., 2017, Nat. Commun. 8, 105), DRUG-seq (Ye et al., 2018, Nat. Commun. 9, 1-9), 3′POOL-seq (Sholder et al., 2020, BMC Genomics 21, 64), PME-seq (Pandey et al., 2020, Nat. Protoc., 15, 1459-1483) and BRB-seq (Alpern et al., 2019 Genome Biol. 20, 71). These techniques have two main commonalities: i) using barcoded DNA oligos used to “tag” poly-adenylated RNA molecules during first strand synthesis and ii) pooling together of all the tagged samples in one tube after the barcoding step.

The overarching goal of these techniques is to decrease the costs and increase the throughput associated with mRNA sequencing library preparation of bulk samples.

This is achieved by reducing reagents, consumables and personnel time through pooling in one solution several barcoded samples. In simple terms, it is much more cost-effective and simpler to process e.g. 100 samples in one tube than 100 samples in 100 tubes.

One of the main challenges of RNA barcoding applied to bulk samples is the ability to guarantee a uniform distribution of sequencing reads across all samples. This challenge is due to the fact that the “molecular barcodes” used during the RNA barcoding step are a functional portion of the “reverse transcription primer” and, as such, different barcodes (i.e. barcodes with different sequences) can have significant effect on the efficiency of the overall workflow. For example, empirical experimental evidence highlights the following potential issues: i) barcodes can lead to unwanted secondary structures that interfere or prevent efficient priming ii) completely random barcodes may end up having very similar or repeating sequences which are then difficult to resolve at the sequencing stage, iii) certain barcodes can be preferentially amplified within the same pool and last, but not least, iv) certain barcodes preferentially bind mitochondrial transcripts, which then appear as an unwanted bias in the sequencing results.

Therefore, there is still a need for more accurate, dependable sequencing tools, (i) and methods that can eliminate barcoding sample-to-sample variation and (ii) methods that use them to improve various barcoding approaches, including the barcoding-mediated high-accuracy sequencing method.

### SUMMARY OF THE INVENTION

The present invention provides one or more oligonucleotide molecules comprising, from 5′ to 3′,
a) a sequencing adapter,
b) a barcode sequence consisting of 9 to 15 nucleotides, preferably 12 nucleotides, and,
c) an mRNA capture sequence.

Further provided is a set of oligonucleotide molecules consisting of 96 oligonucleotide molecules, each molecule comprising, from 5′ to 3′,
a) a sequencing adaptor,
b) a barcode sequence independently selected from the group consisting in SEQ ID: NO. 1 to 96, and,
c) an mRNA capture sequence.

Further provided is a barcode oligonucleotide sequence selected from the group comprising SEQ ID NO: 1 to SEQ ID NO: 96.

Further provided is the use of one or more oligonucleotide molecules of the invention, or of a set of oligonucleotide molecules of the invention, or of a barcode oligonucleotide sequence, or a combination of one or more thereof, of the invention, in a sequencing method.

Further provided is a method for providing a cDNA library, the method comprising the steps of
a) Providing a plurality of RNA samples obtained from a biological sample;
b) Contacting separately each RNA sample with one or more oligonucleotide molecules of the invention, or of a library of the invention, or of a barcode oligonucleotide sequence or a combination of one or more thereof, of the invention, under annealing conditions;
c) Incubating separately each sample under reverse transcription reaction conditions;
d) Pooling together all the cDNA:RNA sample;
e) Proceeding to second strand synthesis under synthesis conditions; and
f) Proceeding with tagmentation and amplification under suitable conditions so as to obtain a cDNA library.

Further provided is a method for sequencing RNA, the method comprising the steps of
a) Providing a cDNA library obtained by the method of claims 9 to 10; and
b) Proceeding to the sequencing under suitable conditions.

Also provided is a method for selecting barcode oligonucleotides for multiplexed nucleic acid sequencing, said method comprising selecting one or more barcode oligonucleotides:
Which has/have a Shannon's first order entropy of at least 1.5 and a second order entropy of at least 2.5; and wherein
GC content of the barcode oligonucleotides is comprised between 35% to 65%;
the first two nucleotides are not G's;
the last two nucleotides are not T's;
the hamming distance between two barcodes of the list should be at least 5; and
said one or more barcode oligonucleotides contain homopolymers of maximum 2 nucleotides.

Also provided is a kit and use of a in a single-cell RNA profiling method.

### DESCRIPTION OF THE FIGURES

FIG. 1 shows a “bad” example of sequencing read distribution for a suboptimal set of 96 barcodes. As can be seen in the circled area, barcodes can systematically underperform as compared to the others.

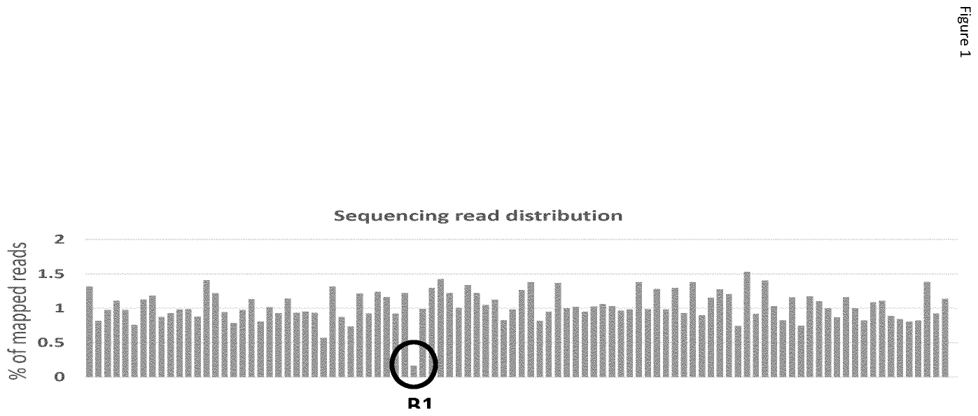

FIG. 2 shows the read distribution of the optimal set of 96 barcodes of the invention in which all barcodes are functional and obtain a similar number of sequencing reads.

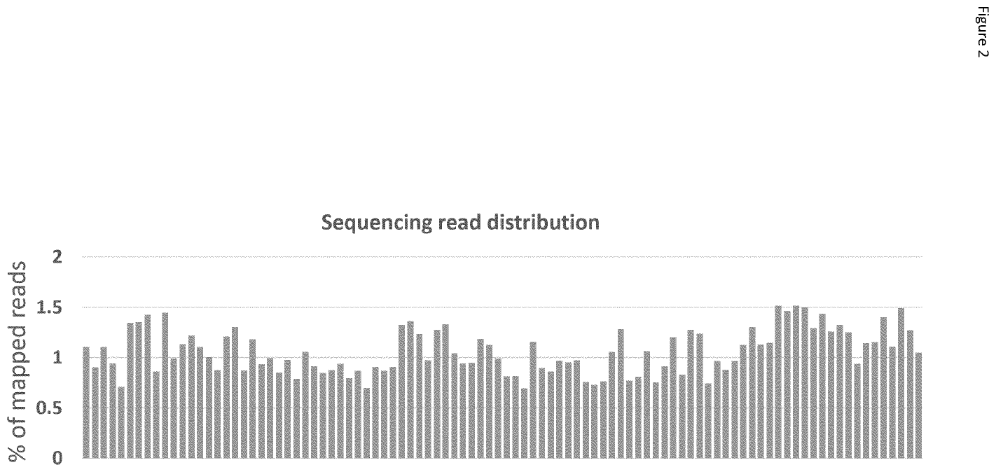

FIG. 3 shows a “bad” example of mitochondrial read distribution for a suboptimal set of 96 barcodes. As can be seen in the circled area, barcodes can systematically obtain more mitochondrial reads as compared to the others.

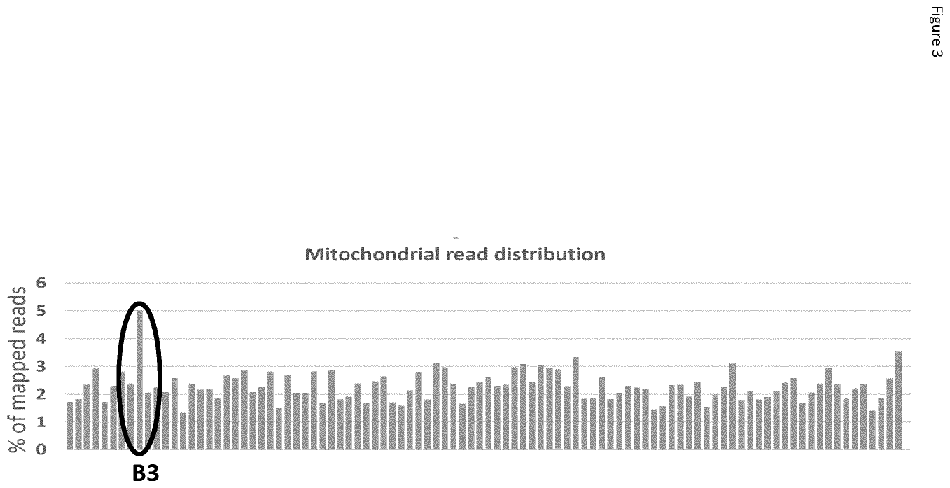

FIG. 4 shows the mitochondrial read distribution of the optimal set of 96 barcodes of the invention in which all barcodes are functional and obtain a similar number of mitochondrial reads.

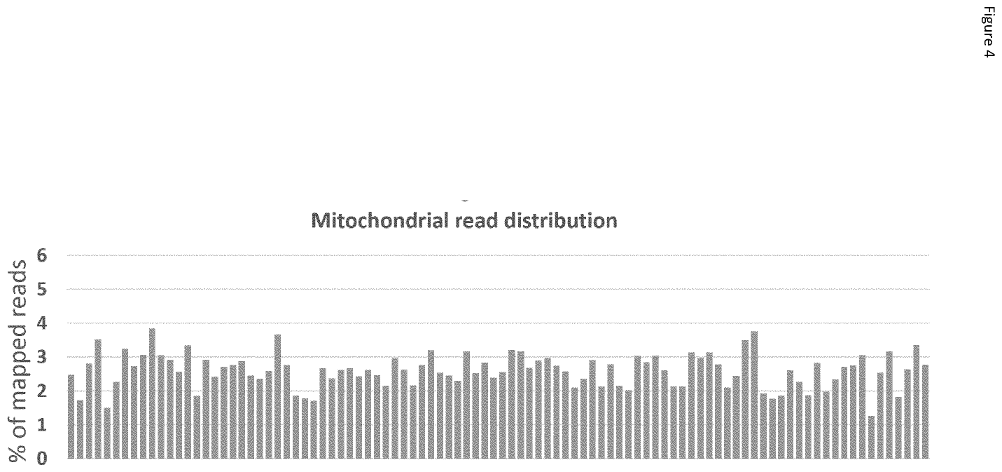

FIG. 5 shows a schematic overview of the method described herein.

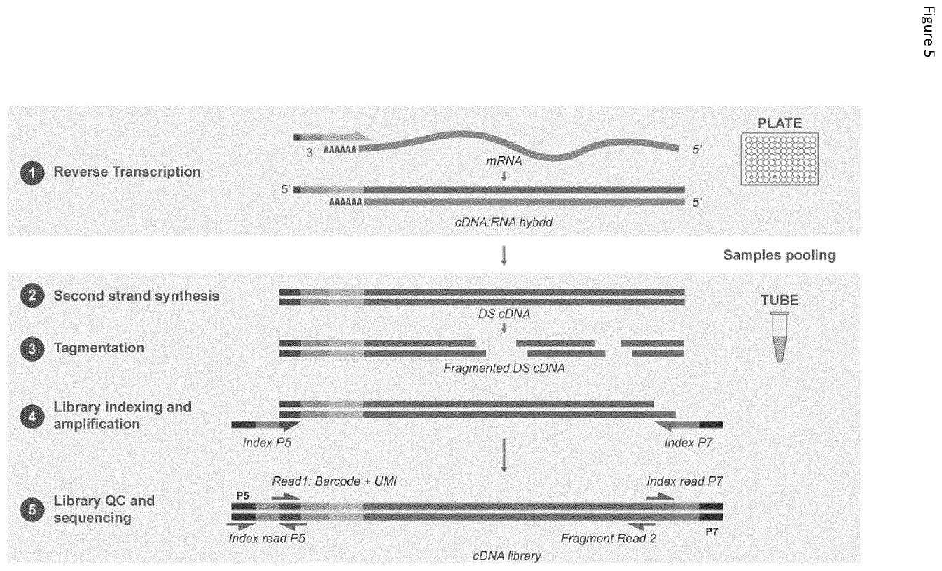

### DESCRIPTION OF THE INVENTION

Although methods and materials similar or equivalent to those described herein can be used in the practice or testing of the present invention, suitable methods and materials are described below. All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

In the case of conflict, the present specification, including definitions, will control. Unless defined otherwise, all technical and scientific terms used herein have the same meaning as is commonly understood by one of skill in art to which the subject matter herein belongs. As used herein, the following definitions are supplied in order to facilitate the understanding of the present invention.

The term “comprise/comprising” is generally used in the sense of include/including, that is to say permitting the presence of one or more features or components. The terms” comprise(s)” and “comprising” also encompass the more restricted ones “consist(s)”, “consisting” as well as “consist/consisting essentially of”, respectively.

As used in the specification and claims, the singular form “a”, “an” and “the” include plural references unless the context clearly dictates otherwise.

As used herein, “one or more” includes” two or more ”, “three or more”, etc. For example, one or more oligonucleotide molecules refers to one oligonucleotide molecule, two oligonucleotide molecules, three oligonucleotide molecules, etc. . . .

The present invention is based on the discovery of an optimal set of 96 barcoded oligonucleotides for multiplexed RNA sequencing. These oligonucleotides contain barcodes that are 12 base pairs long and have been therefore selected from a pool of 4\hat{ }12=16′777′216 potential candidates. This large pool has been filtered twice, first computationally and then experimentally as disclosed herein. The goal was to obtain an optimized set of barcodes for further being able to
1) uniquely demultiplex the samples, with error-tolerance for sequencing errors, and
2) adapt the barcodes for potential technical bias such as overrepresentation of polyT sequences due to preferential amplification of certain sequences.

In one aspect, the invention provides one or more oligonucleotide molecules comprising, from 5′ to 3′,
a) a sequencing adapter,
b) a barcode sequence consisting of 9 to 15 nucleotides, preferably 12 nucleotides, and,
c) an mRNA capture sequence.

In one aspect, the one or more oligonucleotide molecules further comprise a unique molecular identifier (UMI).

“Oligonucleotide” or “polynucleotide,” which are used synonymously, means a linear polymer of natural or modified nucleosidic monomers linked by phosphodiester bonds or analogs thereof. The term “oligonucleotide” usually refers to a shorter polymer, e.g., comprising from about 3 to about 100 monomers, and the term “polynucleotide” usually refers to longer polymers, e.g., comprising from about 100 monomers to many thousands of monomers, e.g., 10,000 monomers, or more. Oligonucleotides and polynucleotides may be natural or synthetic. Oligonucleotides and polynucleotides include deoxyribonucleosides, ribonucleosides, and non-natural analogs thereof, such as anomeric forms thereof, peptide nucleic acids (PNAs), and the like, provided that they are capable of specifically binding to a target genome by way of a regular pattern of monomer-to-monomer interactions, such as Watson-Crick type of base pairing, base stacking, Hoogsteen or reverse Hoogsteen types of base pairing, or the like.

The terms “peptide,” “protein,” and “polypeptide” are used interchangeably to refer to a natural or synthetic molecule comprising two or more amino acids linked by the carboxyl group of one amino acid to the alpha amino group of another.

The term “nucleic acid” refers to a natural or synthetic molecule comprising a single nucleotide or two or more nucleotides linked by a phosphate group at the 3′ position of one nucleotide to the 5′ end of another nucleotide. The nucleic acid is not limited by length, and thus the nucleic acid can include deoxyribonucleic acid (DNA) or ribonucleic acid (RNA). “Sequencing” refers to determining the order of nucleotides (base sequences) in a nucleic acid sample, e.g. DNA or RNA. Many techniques are available such as Sanger sequencing and High Throughput Sequencing technologies (HTS). Sanger sequencing may involve sequencing via detection through (capillary) electrophoresis, in which up to 384 capillaries may be sequence analysed in one run. High throughput sequencing involves the parallel sequencing of thousands or millions or more sequences at once. HTS can be defined as Next Generation sequencing, i.e. techniques based on solid phase pyrosequencing or as Next-Next Generation sequencing based on single nucleotide real time sequencing (SMRT). HTS technologies are available such as offered by Roche, Illumina and Applied Biosystems (Life Technologies). Further high throughput sequencing technologies are described by and/or available from Helicos, Pacific Biosciences, Complete Genomics, Ion Torrent Systems, Oxford Nanopore Technologies, Nabsys, ZS Genetics, GnuBio. Each of these sequencing technologies have their own way of preparing samples prior to the actual sequencing step. Depending on the sequencing technology used, amplification steps may be omitted.

As used herein, the term “barcode” refers to a unique oligonucleotide sequence that allows a corresponding nucleic acid base and/or nucleic acid sequence to be identified. In certain aspects, the nucleic acid base and/or nucleic acid sequence is located at a specific position on a larger polynucleotide sequence (e.g., a polynucleotide covalently attached to a bead). In certain aspects, barcodes can each have a length within a range of from 4 to 150 nucleotides. The barcode technology (or barcoding) has been a particularly powerful technique for studying the genetic and functional variations of the target pool and for high-accuracy target DNA sequencing. Each barcode can comprise deoxyribonucleotides, optionally all of the nucleotides in a barcode region are deoxyribonucleotides. One or more of the deoxyribonucleotides may be a modified deoxyribonucleotide (e.g. a deoxyribonucleotide modified with a biotin moiety or a deoxyuracil nucleotide). The barcodes may comprise one or more degenerate nucleotides or sequences. The barcode regions may not comprise any degenerate nucleotides or sequences.

In one aspect, the barcode sequence of the invention consists of 9 to 15 nucleotides, preferably 12 nucleotides. More preferably, the barcode sequence is selected from the group comprising SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13, SEQ ID NO. 14, SEQ ID NO. 15, SEQ ID NO. 16, SEQ ID NO. 17, SEQ ID NO. 18, SEQ ID NO. 19, SEQ ID NO. 20, SEQ ID NO. 21, SEQ ID NO. 22, SEQ ID NO. 23, SEQ ID NO. 24, SEQ ID NO. 25, SEQ ID NO. 26, SEQ ID NO. 27, SEQ ID NO. 28, SEQ ID NO. 29, SEQ ID NO. 30, SEQ ID NO. 31, SEQ ID NO. 32, SEQ ID NO. 33, SEQ ID NO. 34, SEQ ID NO. 35, SEQ ID NO. 36, SEQ ID NO. 37, SEQ ID NO. 38, SEQ ID NO. 39, SEQ ID NO. 40, SEQ ID NO. 41, SEQ ID NO. 42, SEQ ID NO. 43, SEQ ID NO. 44, SEQ ID NO. 45, SEQ ID NO. 46, SEQ ID NO. 47, SEQ ID NO. 48, SEQ ID NO. 49, SEQ ID NO. 50, SEQ ID NO. 51, SEQ ID NO. 52, SEQ ID NO. 53, SEQ ID NO. 54, SEQ ID NO. 55, SEQ ID NO. 56, SEQ ID NO. 57, SEQ ID NO. 58, SEQ ID NO. 59, SEQ ID NO. 60, SEQ ID NO. 61, SEQ ID NO. 62, SEQ ID NO. 63, SEQ ID NO. 64, SEQ ID NO. 65, SEQ ID NO. 66, SEQ ID NO. 67, SEQ ID NO. 68, SEQ ID NO. 69, SEQ ID NO. 70, SEQ ID NO. 71, SEQ ID NO. 72, SEQ ID NO. 73, SEQ ID NO. 74, SEQ ID NO. 75, SEQ ID NO. 76, SEQ ID NO. 77, SEQ ID NO. 78, SEQ ID NO. 79, SEQ ID NO. 80, SEQ ID NO. 81, SEQ ID NO. 82, SEQ ID NO. 83, SEQ ID NO. 84, SEQ ID NO. 85, SEQ ID NO. 86, SEQ ID NO. 87, SEQ ID NO. 88, SEQ ID NO. 89, SEQ ID NO. 90, SEQ ID NO. 91, SEQ ID NO. 92, SEQ ID NO. 93, SEQ ID NO. 94, SEQ ID NO. 95, SEQ ID NO. 96, or a combination of one or more thereof.

As used herein, the term “sequencing adapter” refers an oligonucleotide sequence that can be used in subsequent sequencing steps (so-called sequencing adapters). Or primers that are used to amplify a subset of fragments prior to sequencing may contain parts within their sequence that introduce sections that can later be used in the sequencing step, for instance by introducing through an amplification step a sequencing adapter or a capturing moiety in an amplicon that can be used in a subsequent sequencing step. Depending also on the sequencing technology used, amplification steps may be omitted.

Any commercially available sequencing adapter can be used, in one aspect, the sequencing adapter comprises, or consists of, CTA CAC GAC GCT CTT CCG ATC (SEQ ID No. 97).

As used herein, a “unique molecular identifier” or UMI is a complex indices added to sequencing libraries before any PCR amplification steps, enabling the accurate bioinformatic identification of PCR duplicates thus enabling to remove PCR duplicates. In one aspect, the UMI is an oligonucleotide sequence consisting of a sequence (N) n (V) m, wherein N is any nucleotide selected from A, T, C and G; V is any nucleotide selected from A, C and G; n is an integer selected from 1 to 20, and m is an integer selected from 1 to 20. Preferably, the UMI comprises, or consists of, SEQ ID NO. 98 (NNNNNNNNNNNVVVVV).

As used herein, an “mRNA capture sequence” is an oligonucleotide sequence that specifically hybridizes to mRNAs. In one aspect, the mRNA capture sequence is a poly-T sequence (10 to 40 T). In a preferred aspect, the mRNA capture sequence is a poly-T sequence (e.g. comprising 10 to 40 T) followed by at least one V and one N, wherein N is any nucleotide selected from A, T, C and G and V is any nucleotide selected from A, C and G. Preferably, the UMI comprises, or consists of, SEQ ID NO. 99 (TTTTTTTTTTTTTTTTTTTTTTTTTTTTTTVN).

“Multiplex sequencing” refers to a sequencing technique that allows for processing a large number of samples on a high-throughput instrument. For multiplex sequencing, individual “barcode” sequences of the invention are added to each sample so that nucleotide sequences from different samples can be distinguished by the unique barcode sequences embedded in each sample. With this technique, multiple DNA or RNA samples can be pooled, processed, sequenced, and analyzed simultaneously.

The present invention further provides a set of oligonucleotide molecules consisting of 96 oligonucleotide, each molecule comprising, from 5′ to 3′,
a) a sequencing adaptor,
b) a barcode sequence independently selected from the group consisting in SEQ ID: NO. 1 to 96,
and,
c) an mRNA capture sequence, as described herein.

In one aspect, the set of oligonucleotide molecules consisting of 96 oligonucleotides further comprises a UMI.

Also provided is the use of one or more oligonucleotide molecules of the invention, or of a set of oligonucleotide molecules of the invention, or of a barcode oligonucleotide sequence, or a combination of one or more thereof, of the invention, in a sequencing method.

The one or more oligonucleotide molecules of the invention, or of a set of oligonucleotide molecules of the invention, or of a barcode oligonucleotide sequence, or a combination of one or more thereof, may be linked by attachment to a solid support (e.g. a bead). A solution of soluble beads (e.g. superparamagnetic beads or styrofoam beads) may be functionalized to enable attachment of two or more oligonucleotide molecules of the invention, set of oligonucleotide molecules of the invention, barcode oligonucleotide sequences, or a combination of one or more thereof. This functionalization may be enabled through chemical moieties (e.g. carboxylated groups), and/or protein-based adapters (e.g. streptavidin) on the beads. The functionalized beads may be brought into contact with a solution of the above-described molecules under conditions which promote the attachment of two or more molecules to each bead in the solution. Optionally, the molecules are attached through a covalent linkage, or through a (stable) non-covalent linkage such as a streptavidin-biotin bond, or a (stable) oligonucleotide hybridization bond.

The present invention further encompasses a method for providing a cDNA library, the method comprising the step of:

Providing a plurality of RNA samples obtained from a biological sample (step a).

As used herein, the term “biological sample” refers to a tissue (e.g., tissue biopsy), organ, cell (including a cell maintained in culture), cell lysate (or lysate fraction), biomolecule derived from a cell or cellular material (e.g. a polypeptide or nucleic acid), or body fluid from a subject. Non-limiting examples of body fluids include blood, urine, plasma, serum, tears, lymph, bile, cerebrospinal fluid, interstitial fluid, aqueous or vitreous humor, colostrum, sputum, amniotic fluid, saliva, anal and vaginal secretions, perspiration, semen, transudate, exudate, and synovial fluid.

The RNA samples can be obtained from any techniques know in the art. In one aspect, the RNA samples According to a particular aspect, the RNA samples are mRNA samples that can be cell lysates, total DNA/RNA eluate, blood and FFPE tissues.

The method further comprises a step (b) of contacting separately each RNA sample with one or more oligonucleotide molecules of the invention, or of a library of the invention, or of a barcode oligonucleotide sequence or a combination of one or more thereof, of the invention, under annealing conditions.

For examples, RNA samples are thawed on ice, transferred to the corresponding wells of the Oligo-dT primer plat, the plate is then sealed with the AluSeal and placed it in a thermocycler at 65° C. for 5 min and immediately put on ice.

The method further comprises a step (c) of incubating separately each sample under reverse transcription reaction conditions.

For example, the RT reaction mix is prepared according to commercial manufacture instruction. Any RT enzyme and buffer commercially available can be used, such as e.g. Lucigen's ERT12910K, ThermoFisher's 18064014 and NEB's M0368S among others.

For example:

| RT Mix | Per well (μL) |
|---|---|
| RT reaction buffer | 5.0 |
| RT reaction enzyme | 0.4 |
| ddH2O | 4.6 |
| TOTAL | 10.0 |

Incubate RT reaction mix in thermocycler with the following program:

| Step | Temperature, ° C. | Time |
|---|---|---|
| Incubation | 42 | 50 min |
| Inactivation | 70 | 10 min |
| Keep | 4 | pause |

The method further comprises a step (d) of pooling together all the cDNA:RNA sample.

The method further comprises a step (e) of proceeding to second strand synthesis under synthesis conditions such as. This second strand synthesis can be generated by any method known in the art. In one aspect, the second strand synthesis method is selected from the group comprising PCR amplification and nick translation, or a combination thereof.

The method further comprises a step (f) of proceeding with tagmentation, and/or end-repair and ligation and amplification under suitable conditions such as, e.g. the conditions described in the examples, so as to obtain a cDNA library.

The term “biological sample” refers to a tissue (e.g., tissue biopsy), organ, cell (including a cell maintained in culture), cell lysate (or lysate fraction), biomolecule derived from a cell or cellular material (e.g. a polypeptide or nucleic acid), or body fluid from a subject. Non-limiting examples of body fluids include blood, urine, plasma, serum, tears, lymph, bile, cerebrospinal fluid, interstitial fluid, aqueous or vitreous humor, colostrum, sputum, amniotic fluid, saliva, anal and vaginal secretions, perspiration, semen, transudate, exudate, and synovial fluid.

Examples of RNAs include but are not limited to: mRNA, amplicons, rRNA, tRNA, nRNA, siRNA, snRNA, snoRNA, scaRNA, microRNA, dsRNA, ncRNA (e.g. IncRNA), ribozyme, riboswitch and viral RNA (e.g., retroviral RNA).

The present invention further encompasses a method for sequencing RNA, the method comprising the steps of
a) Providing a cDNA library obtained by the method described herein; and
b) Proceeding to the sequencing under suitable conditions such as, e.g. those defined by NGS sequencing providers, which are also known in the art.

The present invention further encompasses a method for selecting barcode oligonucleotides for multiplexed nucleic acid sequencing, said method comprising selecting one or more barcode oligonucleotides
Which has/have a Shannon's first order entropy of at least 1.5 and a second order entropy of at least 2.5; and wherein
GC content of the barcode oligonucleotides is comprised between 35% to 65%;
the first two nucleotides are not G's;
the last two nucleotides are not T's;
the hamming distance between two barcodes of the list should be at least 5; and
said one or more barcode oligonucleotides contain homopolymers of maximum 2 nucleotides.

The present method is aimed at enhancing the complexity of the barcode sequences, avoiding having barcodes with repetitive patterns, and reducing internal hairpin propensity.

Moreover, guanidine bases at the beginning of the barcode were removed to avoid the GGC and GGT Illumina sequencing patterns, which are known combinations of nucleotide which are prone to signal to noise decline during sequencing.

Also, thymine bases at the 3′ end were removed to avoid preferential selection/amplification of these barcodes.

Finally, hamming distance between two barcodes of the list was set to at least 5 the enhance the efficacy of later demultiplexing of these barcodes and especially to be able to correct for potential sequencing errors in the barcodes.

Examples of nucleic acids include but are not limited to: DNA, RNA, amplicons, cDNA, dsDNA, ssDNA, plasmid DNA, cosmid DNA, high Molecular Weight (MW) DNA, chromosomal DNA, genomic DNA, viral DNA, bacterial DNA, mtDNA (mitochondrial DNA), mRNA, IRNA, tRNA, nRNA, siRNA, snRNA, snoRNA, scaRNA, microRNA, dsRNA, ribozyme, riboswitch and viral RNA (e.g., retroviral RNA). Preferably, the nucleic acid is an RNA selected form the group comprising mRNA, amplicons, IRNA, tRNA, nRNA, siRNA, snRNA, snoRNA, scaRNA, microRNA, dsRNA, ncRNA (e.g. IncRNA), ribozyme, riboswitch and viral RNA (e.g., retroviral RNA).

In one aspect, the method for selecting barcode oligonucleotides for multiplexed nucleic acid sequencing provided herein is a computer implemented method.

The one or more oligonucleotide molecules of the invention may be linked by attachment to a solid support (e.g. a bead).

Also contemplated is one or more kits for performing one or more methods according to the invention. The one or more kit comprising
i) a set of oligonucleotide molecules consisting of 96 oligonucleotide molecules, each molecule comprising, from 5′ to 3′, a) a sequencing adaptor, b) a barcode sequence independently selected from the group consisting in SEQ ID: 1 to 96, c) optionally a UMI and, d) an mRNA capture sequence, a support for, such as a 96-well plate, and
ii) reagents for sequencing.

The kit can comprise various molecular biology reagents, including DNA polymerases, RNA polymerases, Reverse-transcriptases, DNA ligases, RNA ligases, transposases, viral integrase, CRISPR/Cas9, zinc finger nucleases, transcription activator-like effector nucleases, exonucleases, endonucleases, Polynucleotide Kinases, nucleotides, oligonucleotides, modified oligonucleotides and optimized buffers.

Further contemplated is the use of the kit of the invention, or of the one or more oligonucleotide molecules of the invention, or of a library of the invention, or of a barcode oligonucleotide sequence or a combination of one or more thereof, of the invention, in a single-cell RNA profiling method. Preferably, the single-cell RNA profiling method is similar to the the Bulk RNA Barcoding and sequencing (BRB-seq) method described in Alpern et al., 2019 Genome Biol. 20, 71.

Those skilled in the art will appreciate that the invention described herein is susceptible to variations and modifications other than those specifically described. It is to be understood that the invention includes all such variations and modifications without departing from the spirit or essential characteristics thereof. The invention also includes all of the steps, features, compositions and compounds referred to or indicated in this specification, individually or collectively, and any and all combinations or any two or more of said steps or features. The present disclosure is therefore to be considered as in all aspects illustrated and not restrictive, the scope of the invention being indicated by the appended Claims, and all changes which come within the meaning and range of equivalency are intended to be embraced therein. Various references are cited throughout this Specification, each of which is incorporated herein by reference in its entirety. The foregoing description will be more fully understood with reference to the following Examples.

### EXAMPLES

### Second-Strand Synthesis

Double-stranded cDNA was generated by either PCR amplification (indicated as PCR in the text) or nick translation (indicated as SSS in the text) [24]. The PCR was performed in 50 μL total reaction volume using 20 μL of pooled and Exol-treated first-strand reaction, 1 μL of 10 μM LA_oligo (Microsynth) primer, 1 μL of dNTP (0.2 mM), 1 μl of with Advantage 2 Polymerase Mix (Clontech, #639206), 5 μl of Advantage 2 PCR buffer, and 22 μL of water following the program (95° C.—1 min; 10 cycles: 95° C.—15 s, 65° C.—30 s, 68° C.—6 min; final elongation at 72° C.—10 min). Alternatively, the second stand was synthesized following the nick translation method. For that, a mix containing 2 μL of RNAse H (NEB. #M0297S), 1 μL of Escherichia coli DNA ligase (NEB, #M0205 L), 5 μL of E. coli DNA Polymerase (NEB, #M0209 L), 1 μL of dNTP (0.2 mM), 10 μL of 5×Second Stand Buffer (100 mM Tris-HCl (pH 6.9) (AppliChem, #A3452); 25 mM MgCl2 (Sigma, #M2670); 450 mM KCl

(AppliChem. #A2939); 0.8 mM β-NAD; 60 mM (NH4)2SO4 (Fisher Scientific Acros, #AC20587); and 11 μL of water was added to 20 μl of Exol-treated first-strand reaction on ice. The reaction was incubated at 16° C. for 2.5 h or overnight. Full-length double-stranded cDNA was purified with 30 μL (0.6x) of AMPure XP magnetic beads (Beckman Coulter, #A63881) and eluted in 20 μL of water.

### Library Preparation and Sequencing

The sequencing libraries were prepared by tagmentation of 1-50 ng of full-length double-stranded cDNA. Tagmentation was done either with Illumina Nextera XT kit (Illumina, #FC-131-1024) following the manufacturer's recommendations or with in-house produced Tn5 preloaded with dual (Tn5-A/B) or same adapters (Tn5-B/B) under the following conditions: 1 μL (11 μM) Tn5, 4 μL of 5×TAPS buffer (50 mM TAPS (Sigma, #TS130), and 25 mM MgCl2 (Sigma, #M2670)) in 20 μl total volume. The reaction was incubated 10 min at 55° C. followed by purification with DNA Clean & Concentrator-5 kit (Zymo Research) and elution in 21 μL of water. After that, tagmented library (20 μL) was PCR amplified using 25 μL NEBNext High-Fidelity 2X PCR Master Mix (NEB, #M0541 L), 2.5 μL of PS_BRB primer (5 μM, Microsynth), and 2.5 μL of oligo bearing Illumina index (Idx7N5 5 μM, IDT) using the following program: incubation 72 ° C—3 min. denaturation 98° C.—30 s; 10 cycles: 98° C.—10 s, 63° C.—30 s, 72° C.—30 s; final elongation at 72° C.—5 min. The fragments ranging 200-1000 bp were size-selected using AMPure beads (Beckman Coulter, #A63881) (first round 0.5% beads, second 0.7×). The libraries were profiled with High Sensitivity NGS Fragment Analysis Kit (Advanced Analytical, #DNF-474) and measured with Qubit dsDNA HS Assay Kit (Invitrogen, #Q32851) prior to pooling and sequencing using the Illumina NextSeq 500 platform using a custom ReadOne primer (IDT) and the High Output v2 kit (75 cycles) (Illumina, #FC-404-2005) The library loading concentration was 2.2 pM. The read1 sequencing was performed for 6-21 cycles and read2 for 54-70 cycles depending on the experiment.

1. One or more oligonucleotide molecules comprising, from 5′ to 3′,
a) a sequencing adapter,
b) a barcode sequence consisting of 9 to 15 nucleotides,
and,
c) an mRNA capture sequence.

2. The one or more oligonucleotide molecules of claim 1, further comprising d) a unique molecular identifier (UMI).

3. The one or more oligonucleotide molecules of claim 1, wherein the sequencing adapter comprises SEQ ID NO: 97.

4. The one or more oligonucleotide molecules of claim 1, wherein the barcode sequence is selected from the group consisting of SEQ ID NO: 1 to SEQ ID NO: 96 and a combination of one or more thereof.

5. The one or more oligonucleotide molecules of claim 2, wherein the UMI consists of a sequence (N)n(V)m, wherein N is any nucleotide selected from A, T, C and G; V is any nucleotide selected from A, C and G; n is an integer selected from 1 to 20, and m is an integer selected from 1 to 20.

6. The one or more oligonucleotide molecules of claim 1, wherein the mRNA capture sequence is a poly-T sequence followed by at least one V and one N, wherein N is any nucleotide selected from A, T, C and G and V is any nucleotide selected from A, C and G.

7. A set of oligonucleotide molecules consisting of 96 oligonucleotide molecules, each molecule comprising, from 5′ to 3′,
a) a sequencing adaptor,
b) a barcode sequence independently selected from the group consisting of SEQ ID NO: 1 to SEQ ID NO: 96,
c) an mRNA capture sequence and,
d) optionally a UMI.

8. A barcode oligonucleotide having a sequence selected from the group consisting of SEQ ID NO: 1 to SEQ ID NO: 96 and a combination of one of more thereof.

9. (canceled)

10. A method for making a cDNA library, the method comprising:
a) Providing a plurality of RNA samples obtained from a biological sample;
b) Contacting separately each RNA sample with one or more oligonucleotide molecules of claim 1 under annealing conditions;
c) Incubating separately each sample under reverse transcription reaction conditions;
d) Pooling together all the cDNA:RNA sample;
e) Proceeding to second strand synthesis under synthesis conditions; and
f) Proceeding with tagmentation and/or end-repair and ligation and amplification under suitable conditions so as to obtain a cDNA library.

11. The method for making a cDNA library of claim 10, wherein the second strand synthesis is generated by a method comprising PCR amplification, nick translation, or a combination thereof.

12. A method for sequencing RNA, the method comprising the steps of
a) Providing a cDNA library obtained by the method of claim 10; and
b) Proceeding to sequencing under suitable conditions.

13. A method for selecting barcode oligonucleotides for multiplexed nucleic acid sequencing, the method comprising selecting one or more barcode oligonucleotides which has/have a Shannon's first order entropy of at least 1.5 and a second order entropy of at least 2.5; and wherein
GC content of the barcode oligonucleotides is comprised between 35% to 65%;
the first two nucleotides are not G's;
the last two nucleotides are not T's;
the hamming distance between two barcodes of the list is at least 5; and
said one or more barcode oligonucleotides contain homopolymers of maximum 2 nucleotides.

14. The method of claim 13, wherein the method is a computer implemented method.

15. A kit comprising:
i) one or more oligonucleotide molecules of claim 1,
ii) a support for sample preparation, and
iii) reagents for sequencing.

16. (canceled)

17. The one or more oligonucleotide molecules of claim 1, wherein the barcode sequence consists of 12 nucleotides.

18. The one or more oligonucleotide molecules of claim 3, wherein the sequencing adaptor consists of SEQ ID NO: 97.
